(19) United States
(12) Patent Application Publication
(10) Pub. No.: US 2002/0015960 A1
(43) Pub. Date: Feb. 7, 2002

(54) Method of preparing nucleic acid microchips

(76) Inventors: Zicai Liang, Sundbyberg (SE); Anil Kumar, Farsta (SE)

Correspondence Address: JENKENS & GILCHRIST, PC, 1445 ROSS AVENUE, SUITE 3200, DALLAS, TX, 75202, US

(21) Appl. No.: 09/909,333

(22) Filed: Jul. 19, 2001

Related U.S. Application Data
(60) Provisional application No. 60/219,376, filed on Jul. 19, 2000.

(51) Int. Cl. C12Q 1/68; C12M 1/34

(52) U.S. Cl. 435/6; 435/287.2; 438/1

(57) ABSTRACT

A method of preparing microchips having nucleic acid attached thereto is disclosed. In the method a surface of a first chip (master chip), to which surface nucleic acid is attached, and a surface of a second chip (print chip), are brought into contact with each other, whereby the nucleic acid attached to the first chip is partially transferred to the surface of the second chip, through detachment from the first chip, and immobilization onto the second chip.

### PRIORITY CLAIM

This Application for Patent claims the benefit of priority from, and hereby incorporates by reference the entire disclosure of, U.S. Provisional Application for Patent Ser. No. 60/219,376 filed Jul. 19, 2000.

### FIELD OF THE INVENTION

The present invention relates to a method of producing nucleic acid microchips, and microchips obtained by the method. More particularly, microchips are prepared by the duplication of a master-chip, thereby obtaining a number of copies of the master chip.

### BACKGROUND OF THE INVENTION

Nucleic acid microchips (or micro-arrays) have rapidly evolved to become one of the essential tools for life science research, ranging over monitoring gem expression, polymorphism analysis, disease screening and diagnostics, nucleic acid sequencing, and genome analysis. They are widely anticipated to be one of the major players in clinical diagnostics and drug development in the post-genome era. Most commercially available microchips are fabricated by high-speed robotics, generally on glass but sometimes on nylon substrates. The terms “microchip” and “chip” as used herein, are intended to be synonymous with one another.

Currently, nucleic acid microchips are produced by methods which fall into two categories, which involve the contacting and attachment of nucleic acid molecules, usually deoxyribonucleic acid (DNA), on the surface of the chip. The first method involves the physical deposition of prefabricated DNA molecules onto microchips, and the second method involves on-chip synthesis of DNA molecules (mostly short oligonucleotides) by either photolithographic synthesis or by piezoelectric printing. The methods of the first category enjoy the advantage of lower cost, and higher flexibility, but suffer from the fact that they are ill-suited for making a very high density chip, i.e., a chip comprising a large number of nucleic acid molecules on its surface. The methods of the second category enable the production of very high-density chips, but the enormous set-up cost for making these chips is a serious limiting factor. Furthermore, the speed of making microchips with any of the currently available methods in the art, is relatively slow. For example, the making of a 5000 DNA spots/chip microchip requires several hours to be completed. Consequently, the cost of chips thus produced has posed serious restraints on the widespread application of nucleic acid microchips.

The aim of the present invention is to provide a new method of nucleic acid chip manufacture that has the potential to increase the current speed of manufacture, and reduce the cost for each chip thus obtained. The invention presented here discloses and claims a novel category of methods of nucleic acid chip production that can afford higher production speed at lower cost

### SUMMARY OF THE INVENTION

A method of making nucleic acid chips is disclosed, namely “chemical nanoprinting”, which method makes it possible to obtain multiple print chips from a single “master-chip”, with each duplicate (copy) chip printed in less than a minute, i.e., about 1,000 times faster than the current methods in the art. High density prints can readily be produced or reproduced, when a high density master-chip is used. The prints obtained by the present method will be essentially identical to the mirror image of the master-chip used. The reproducibility of this method, with respect to the geometric shape and the distribution of the printed pattern obtained therewith, is thus better than for any currently known alternative technique in the art. This procedure has the potential to be combined with the in situ synthesis or physical deposition methods currently known in the art, to increase the overall throughput of nucleic acid chip production by a factor of 10-100.

This technology allows the direct printing of chips with a wide range of densities in a short period of time, and more importantly, has the potential to make several printout chips from a single master-chip.

### BRIEF DESCRIPTION OF DRAWINGS

FIGS. 1A-1C shows a master-chip and two copies thereof, having oligonucleotides transferred from the master-chip attached to an acrylamide layer.

FIGS. 2A-2F shows two master-chips, 1 and 2, before and after printing, respectively, and prints obtained from the latter master-chip at different temperatures.

In FIG. 3A-3K, a master-chip and 10 print-chips are shown, obtained from the master-chip using varying length of heating time.

In FIG. 4, the hybridization signals of the 10 print chips and the master chip after printing are compared.

FIGS. 5A and 5B shows a high density master-chip and print chip obtained therefrom.

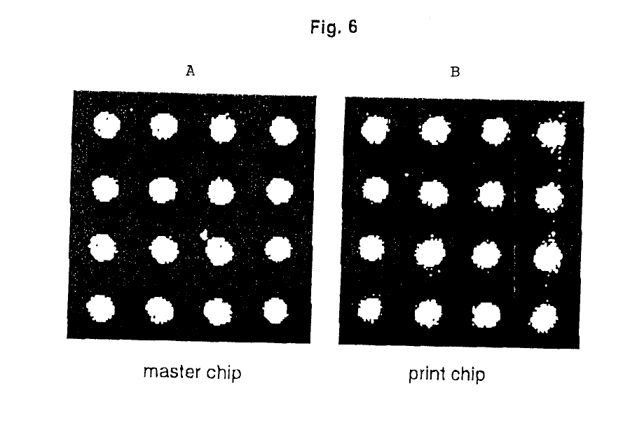

In FIG. 6A and 6B, enlarged views of the chips from FIG. 5 are shown.

### DETAILED DESCRIPTION OF THE INVENTION

The method of the present invention is based on partial detachment of nucleic acid molecules attached to a first chip, the master-chip, and subsequent binding of said detached nucleic acid molecules to a second chip, the print or copy chip, when the second chip is brought into physical contact with the master-chip.

The nature of the detachment and bonding are chemical in nature. Nucleic acid chips are fabricated by high-speed robotics, on glass or nylon substrates. The nucleic acid molecules are tethered onto the chip surface via disulphide linkages. The present inventors have discovered that acrylamide has a limited reactivity towards these disulphide linkages. The acrylamide causes the detachment of nucleic acid molecules from a solid support, followed by the simultaneous conjugation of these nucleic acid molecules with the acrylamide.

It has now been found that one characteristic of the interaction between the acrylamide and the disulphide bonds is that the reaction only results in partial displacement of the nucleic acid molecules from the original chip (alternately referred to herein as the “master chip”). This fact has been demonstrated by overlaying a nucleic acid chip with a layer of a mixture of acrylamide bis-acrylamide in the ratio of 20:1, and triggering the polymerization by means of TEMED (N,N,N′,N′-Tetramethylethylene-diamine) and ammonium persulfate, followed by heating of the chip-gel complex at 95-100° C. for at least one minute.

The covalent nature of the linkage of Lac-acrylic to (3-mercaptopropyl) trimethoxysilane has been confirmed by repeated stripping by boiling in water. This result indicates that the acrylic group has a limited level of reactivity towards thiol as well as disulphide groups. The thiol groups in mercaptosilane molecules can undergo spontaneous oxidation to form disulphide bonds. Although it is not a major focus for the purpose of this disclosure to distinguish the two states of the mercaptosilane, the present inventors suspect that the reactivity of the acrylic groups is towards the disulphide bonds.

In an embodiment of the invention, the nucleic acid molecules are transferred from the master-chip to the print (or copy) chip, by bringing the surface of the master chip in contact with the surface layer of the print chip. In order to facilitate printing, the surface of the print chip may be in a relatively liquid state. In an alternate embodiment of the invention, the surface of the chip to be printed, i.e., print chip, may be a rubber material, which is compressible, thereby negating the need for the print chip surface to be in a liquid state. In an embodiment of the invention, the surface of the print chip may comprise a layer of acrylamide.

Accordingly, the present inventors have found that, by loading the original or master chip with a relatively high quantity of nucleic acid molecules, the nucleic acid molecules can be “printed” onto an acrylamide layer through the partial detachment process discussed above, which is caused by the interaction between the acrylic groups of the acrylamide layer and the disulphide groups.

In an embodiment of the invention, the following criteria are followed:

a) the master-chip is preferably able to harbor sufficient amount of nucleic acid molecules for multiple printing, i.e., more than 2 prints;

b) the transfer of nucleic acid molecules from the master chip to the print chip is performed in a controlled manner, so that each print chip will be configured to have relatively the same surface concentration of nucleic acid molecules;

c) the printing is preferably performed at high resolution, so that high density chips can be reproduced by this mechanism.

1 nM dots (50 pl×1 nM/0.01mm², equals to 0.05 pmoles/cm²) can be routinely detected with a laser scanner from Genetic Microsytems, and a surface density of 5 pmoles/cm² will give satisfactory hybridization signals. Good deposition methods, such as, for example, photolithographic synthesis or piezoelectric printing can generate nucleic acid chips having a nucleic acid molecule surface density of at least 50 pmoles/cm² and in situ synthesis can produce nucleic acid chips having a surface density of greater than 2000 pmoles/cm² These numbers suggest that the current state of the art technology can produce master chips which contain relatively high concentration of nucleic acid molecules for printing purposes, and can be used to generate a master chip in an embodiment of the invention.

An embodiment of the invention provides a method for preparing nucleic acid microchips comprising, attaching nucleic acid molecules to a first surface of a first chip, and contacting said first surface of said first chip with a first surface of a second chip. The first surface of the second chip may be in a relatively liquid state. In certain embodiments of the invention, the first surface of the second chip may comprise a rubber material or alternately may comprise an acrylamide layer. In an embodiment of the invention, the nucleic acid molecules attached to the surface of the master chip are deoxyribonucleic acid (DNA). In other embodiments of the invention, the nucleic acid molecules attached to the surface of the first or master chip are ribonucleic acid (RNA).

In an embodiment of the invention, the surface density of nucleic acid molecules on the surface of the master chip is at least 50 pmoles/cm², more preferably ranging from 50-2000 pmoles/cm², and most preferably greater than 2000 pmoles/cm². In an embodiment of the invention, the nucleic acid molecules are attached to the surface of the master chip by disulphide bonds

In an embodiment of the invention, the print chips are generated at an ambient temperature of 25° C. The print chips may also be generated at a temperature range of 25° C. to 100° C. In certain embodiments of the invention, the printing temperature is 95° C., more preferably 99° C., and most preferably 100° C. In other embodiments of the invention, the printing temperature may be at least 30° C.

In an embodiment of the invention, the contact time between the master chip and a second chip in order to generate a print chip, i.e., printing time, varies from about 10 seconds to about 10 minutes. In certain embodiments of the invention, the printing time is at least 10 seconds.

In an embodiment of the invention, the number of print chips generated from a single master chip ranges from about two (2) to about two hundred (200). In certain embodiments of the invention, at least two print chips are generated from a single master ship.

### WORKING EXAMPLE

### EXAMPLE 1

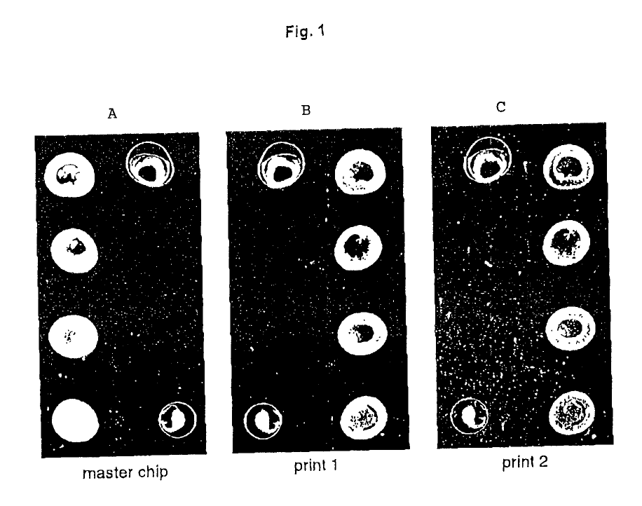

A master chip hand-spotted with DNA oligonucleotides, as shown in FIG. 1, was used to make prints on two acrylamide-coated chips (print 1 and print 2) at a printing temperature of 99° C. All three chips were hybridized to a complementary probe, labeled with Cy3, a water-soluble fluorescent label. As can be seen from FIG. 1, oligonucleotides immobilized on a glass surface via a disulphide bond can be transferred to acrylamide coated chips with great spatial precision.

### EXAMPLE 2

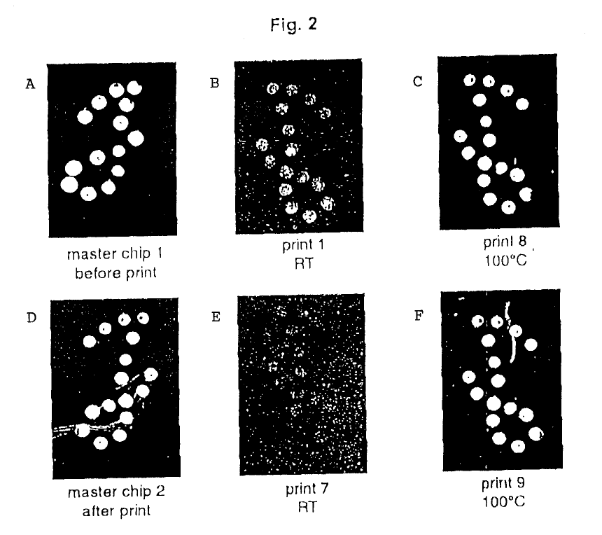

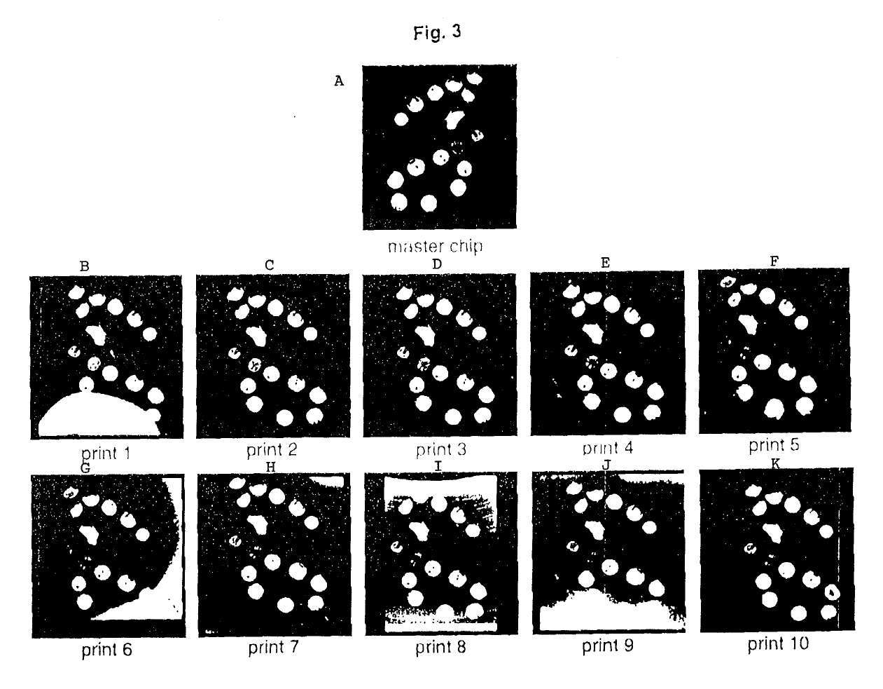

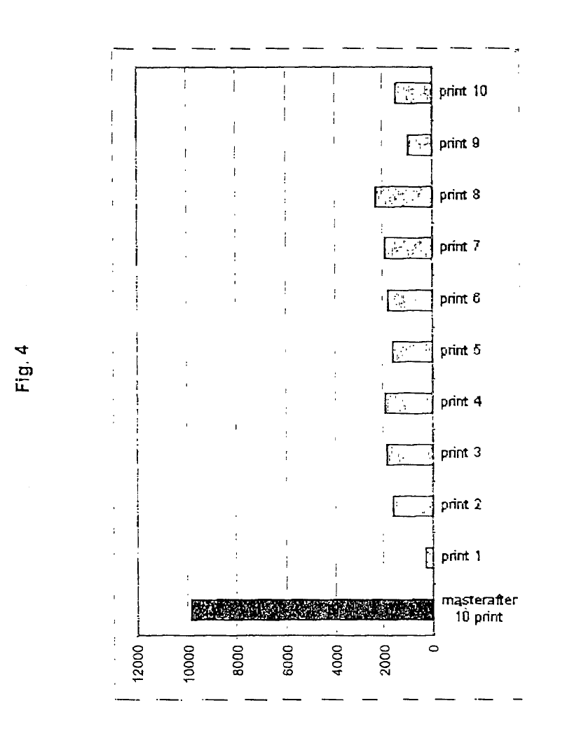

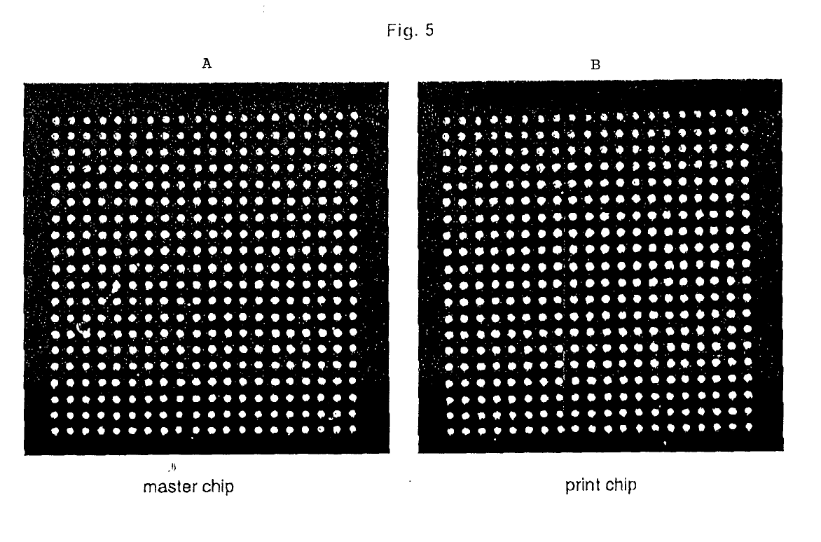

This example was designed for quantitative control of downloading. The contribution of contact time and temperature to the downloading process was analyzed. Seven acrylamide layer prints, 1 to 7, were sequentially generated from a single chip, i.e. master-chip 2, at room temperature (RT; 25° C.) where the gel, and the master-chip were allowed to remain in contact for twenty (20) seconds, forty (40) seconds, one (1) minute, two (2) minutes, three (3) minutes, four (4) minutes and five (5) minutes respectively. As seen in FIG. 2B and FIG. 2E, the hybridization results indicate that there was no significant transfer of oligonucleotides from the master-chip to either print 1 or print 7, with the exception that print 1 seemed to have received a greater amount of oligonucleotides than print 1 or print 7. The somewhat higher DNA transfer of print 1, might be due to oligonucleotides that were non-covalently attached on the glass surface of the master-chip. When generating print 8, the chip-gel complex was placed in water and the water brought to a boil in a microwave (60 seconds in total), before separating the gel monolayer from the master chip 2. In contrast to prints made at room temperature, print 8 shows a strong signal when hybridized to the fluorescent probe, indicating significantly higher levels of oligonucleotides relative to print 1 and print 7. This result was further confirmed by the making of a ninth print from the same master-chip in the same way as for the eighth print, also shown in FIG. 2F. The hybridization signal visualized on print 9 appears to be at similar levels as print 8. These results suggest that heat treatment is essential to trigger the “downloading” or “detachment” of oligonucleotides from master-chip to the acrylamide layer print chips. The transfer of disulphide bond-tethered oligonucleotides from glass or nylon surface of the master chip to the acrylamide surface of the print chip can be controlled by modulating the temperature. Master chip 2 after 9 printings and master chip 1, were also hybridized to Cy3 labeled probes, and are shown in FIGS. 2B and 2A respectively.

### EXAMPLE 3

In theory, about 100-200 prints can be made from best slides generated from the state of the art technology. In order to prove the feasibility on this aspect, multiple copies on acrylamide coated chips were made sequentially from S-S linked oligonucleotide chips at a temperature of 99° C., with the following printing regime: print 1, 10 seconds; print 2, 15 seconds; print 3, 20 seconds; print 4, 30 seconds; print 5, 45 seconds; print 6, 1 minute; print 7, 2 minutes, print 8, 3 minutes; print 9, 4 minutes; and print 10, 10 minutes. Following this step, the prints and the master-chips were all evaluated by hybridization with a Cy3 labeled probe, as shown in FIG. 3. Thus, multiple print-chips can be manufactured by printing from a single glass chip.

In FIG. 4, the hybridization signal of the 9 print-chips and the master chip (after printing) are compared. As can be seen, using S-S linked oligonucleotide chips of this Example as the master-chips, several prints on acrylamide monolayers can be made with similar levels of intensity, i.e., oligonucleotide transfer, with the exception of print 1, suggesting that 10 seconds may not be enough for the printing complex to be heated to a critical temperature.

### EXAMPLE 4

The resolution of the printing was studied by introducing an automated arrayer (Genetic Microsystems) into the process. A temperature of 95° C., and a contact time of 1 min. were used. This arrayer can deposit DNA samples at a density of 1,000 spot/cm² (10,000/chip) with a distance of 375 μm between spots (average volume of liquid delivery is 50 pl/spot). A master chip with 100 μm spots (300 μm pitch) was printed with an acrylamide coated chip. The master and print chip were then hybridized to a Cy3 labeled probe.

With reference to FIG. 5, corresponding regions of the master and print chips were enlarged, and the size of 400 spots on the print chip was compared with that of the master-chip. As evidenced by FIG. 5, only minimal resolution loss can be observed on the print chip. However, when the spots were sufficiently enlarged, a fuzzy edge around the spots on the print chip could be observed, which can amount to an increase of a few μm in diameter per spot. Since the actual oligonucleotide transfer occurs after the polymerization of the acrylamide, it is believed that this increase of the spot size is the result of the locomotion of individual acrylamide fibers in the subsequent handling. As long as the gaps between spots are significantly larger than 20 μm (with a spot density of 1000-50,000 spots/cm², (10,000-400,000/chip), the present inventors believe that the printing resolution is high enough to generate printout chips with acceptable quality. When the spot density reaches even higher, there seems to be the likelihood that spots will start to smear each other on the printout chips. In case of the acrylamide, however, the locomotion of polyacrylamide fibers would be closely related to the degree of crosslinking of the acrylamide gel, and better polymerisation scheme may be able to diminish this “diffusing” effect, thus allowing the skilled person to make prints from chips of much higher spot density.

Other polymers and corresponding chemistry could also be used in the method of the present invention, as long as a sufficient degree of contact between the oligonucleotides and the polymer surface is guaranteed, in order to allow for the detachment and binding to take place.

What is claimed is:

1. A method for preparing nucleic acid microchips comprising:
attaching nucleic acid molecules to a first surface of a first chip, and contacting said first surface of said first chip with a first surface of a second chip.

2. The method of claim 1, wherein the nucleic acid molecules are DNA.

3. The method of claim 1, wherein the nucleic acid molecules are RNA.

4. The method of claim 1, wherein the first surface of the second chip is in a relatively liquid state.

5. The method of claim 1, wherein the first surface of the second chip comprises a rubber material.

6. The method of claim 1, wherein the first surface of the second chip comprises an acrylamide layer.

7. The method of claim 1, wherein the first surface of the first chip comprises a nucleic acid surface density of at least 50 pmoles/cm², more preferably ranging from 50-2000 pmoles/cm², and most preferably greater than 2000 pmoles/cm².

8. The method of claim 1, wherein the nucleic acid molecules are attached to the first surface of the first chip by disulphide bonds.

9. The method of claim 1, wherein the printing temperature is 25° C.

10. The method of claim 1, wherein the printing temperature ranges from 25° C.-100° C.

11. The method of claim 1, wherein the printing temperature is 95° C., more preferably 99° C., and most preferably 100° C.

12. The method of claim 1, wherein the printing temperature is at least 30° C.

13. The method of claim 1, wherein the printing time varies from about 10 seconds to about 10 minutes.

14. The method of claim 1, wherein the printing time is at least 15 seconds.

15. The method of claim 1, wherein the number of print chips generated from a single master chip ranges from 2-200 print chips.

16. The method of claim 1, wherein the number of print chips generated from a single master chip is at least two.

17. The method of claim 1, wherein the nucleic acid is RNA or DNA.

18. A nucleic acid microchip prepared by a method comprising:
attaching nucleic acid molecules to a first surface of a first chip, and contacting said first surface of said first chip with a first surface of a second chip.

19. The microchip of claim 18, wherein the nucleic acid molecules are DNA.

20. The microchip of claim 18, wherein the nucleic acid molecules are RNA.

21. The microchip of claim 18, wherein the first surface of the second chip is in a relatively liquid state.

22. The microchip of claim 18, wherein the first surface of the second chip comprises a rubber material.

23. The microchip of claim 18, wherein the first surface of the second chip comprises an acrylamide layer.

24. The microchip of claim 18, wherein the first surface of the first chip comprises a nucleic acid surface density of at least 50 pmoles/cm², more preferably ranging from 50-2000 pmoles/cm², and most preferably greater than 2000 pmoles/cm².

25. The microchip of claim 18, wherein the nucleic acid molecules are attached to the first surface of the first chip by disulphide bonds.

26. The microchip of claim 18, wherein the printing temperature is 25° C.

27. The microchip of claim 18, wherein the printing temperature ranges from 25° C.-100° C.

28. The microchip of claim 18, wherein the printing temperature is 95° C., more preferably 99° C., and most preferably 100° C.

29. The microchip of claim 18, wherein the printing temperature is at least 30° C.

30. The microchip of claim 18, wherein the printing time varies from about 10 seconds to about 10 minutes.

31. The microchip of claim 18, wherein the printing time is at least 15 seconds.

32. The microchip of claim 18, wherein the number of print chips generated from a single master chip ranges from 2-200 print chips.

33. The microchip of claim 18, wherein the number of print chips generated from a single master chip is at least two

34. The microchip of claim 18, wherein the nucleic acid is RNA or DNA.
